(19) United States
(12) Patent Application Publication
(10) Pub. No.: US 2020/0215118 A1
(43) Pub. Date: Jul. 9, 2020

(54) PHARMACEUTICAL COMPOSITION FOR TREATMENT OF ATOPY CONTAINING EXOSOMES DERIVED FROM NEURAL STEM CELLS

(71) Applicant: KOREA UNIVERSITY RESEARCH AND BUSINESS FOUNDATION, Seoul (KR)

(72) Inventors: Sung Hoi HONG, Seoul (KR); Seulbee LEE, Seoul (KR)

(73) Assignee: KOREA UNIVERSITY RESEARCH AND BUSINESS FOUNDATION, Seoul (KR)

(21) Appl. No.: 16/733,484

(22) Filed: Jan. 3, 2020

(30) Foreign Application Priority Data
Jan. 3, 2019 (KR) 10-2019-0000629

(51) Int. Cl. A61K 35/30; A61P 17/04; A61K 38/50; A61K 38/17

(52) U.S. Cl. CPC A61K 35/30; A61K 38/1761; A61K 38/50; A61P 17/04

(57) ABSTRACT

The present invention relates to a pharmaceutical composition for treating atopy, which contains, as an active ingredient, neural stem cell-derived exosomes or a neural stem cell-conditioned medium containing neural stem cell-derived exosomes, and a method of treating atopy by administering the composition.

### TECHNICAL FIELD

The present invention relates to a pharmaceutical composition for treating atopy, which contains, as an active ingredient, neural stem cell-derived exosomes or a neural stem cell-conditioned medium containing neural stem cell-derived exosomes.

### BACKGROUND ART

Atopic dermatitis is a skin disease caused by environmental pollution due to industrialization, increased use of food additives, exposure to various antigens due to western housing forms, and reduced immune resistance due to excessive hygiene. It is a disease which is caused by environmental factors in most cases and is also attributable to family history and genetic factors, because it has been reported that when if parents suffer from atopic dermatitis, their offspring are very likely to suffer from the same disease, and that genetic mutations occur in the gene FLG encoding the protein filaggrin which forms a skin protective barrier in keratinocytes, causing atopy. It is known that atopy is accompanied by symptoms, including severe itching and lesions such as dry skin and eczema. The distribution of lesions and the pattern of responses differ depending on age, and thus atopy is classified into infant atopy and adult atopy.

If the skin barrier is not smoothly formed due to environmental or genetic factors as described above or continued scratching of an already formed lesion damages the skin barrier, the sensitivity of the skin barrier to specific external antigens increases. As a result, excessive infiltration of dendritic cells presenting antigens occurs, and these cells also secrete mediators, which induce itching, and cytokines and chemokines which trigger immune cells. If these symptoms are repeated, lichenification appears in which the skin becomes thickened.

Therapies that ultimately eliminate the causes of atopic dermatitis have not been developed, and methods for treating atopic dermatitis mostly include the administration of antihistamines for treating itching, the use of immunomodulators or topical or systemic steroids for suppressing excessive immune responses, and the application of moisturizers including ceramides, which are skin barrier forming proteins for protecting damaged skin barriers.

Steroids, which are widely used in the treatment of atopic dermatitis, adversely affect the growth and development of children when used for a long period of time, and have a side effect of causing a more severe immune response if the administration thereof is stopped immediately after alleviation of symptoms. In addition, the use of immunomodulators has a side effect of causing a burning sensation on the skin, and these steroids have disadvantages in that they are difficult to use against chronic diseases such as atopic dermatitis disease for a long period of time and hardly exhibit a perfect therapeutic effect.

Until now, the development of agents for treating atopy has been focused mainly on vitamins for alleviating itching, moisturizers including phospholipids, probiotics for alleviating atopy through improved eating habits, and interleukin inhibitors for inhibiting interleukins that infiltrate immune cells into skin lesions and cause persistent immune response. In recent years, based on the immunosuppressive and immunomodulatory abilities of cord blood, bone marrow and adipose-derived mesenchymal stem cells, studies on therapeutic agents using stem cell-derived products and stem cells themselves have been actively conducted.

The present invention is intended to develop an atopic dermatitis therapeutic agent based on stem cells using extracellular vesicles (exosomes), which exactly reflect the immunosuppressive properties and characteristics of neural stem cells, and a conditioned medium containing the exosomes, by culturing neural stem cells present in the human ventricular zone rather than culturing cord blood, bone marrow and adipose-derived mesenchymal stem cells. Among conventional arts based on the present invention, Korean Patent Publication No. 2014-0024310 discloses that exosomes isolated from a bone marrow-derived mesenchymal stem cell-conditioned medium can be used to treat lung disease, and Korean Patent Publication No. 2015-0108795 showed that the treatment of inflammatory brain disease with stem cell-derived exosomes inhibited brain tissue damage and neuronal death, but did not specify the kind of stem cells. Finally, Korean Patent Publication No. 2013-00116552 discloses that exosomes derived from bone marrow-derived mesenchymal stem cells can be used as a therapeutic agent against nervous disease.

As described above, although technologies of isolating mesenchymal stem cell-derived exosomes, which can be used against inflammatory diseases and are obtained from various adult tissues, have been filed for patent protection, the anti-inflammatory effect or atopy alleviation effect of exosomes obtained from human neural stem cells has not been known yet.

Accordingly, the present inventors have made extensive efforts to isolate exosomes containing a number of anti-inflammatory factors having an atopy inhibitory effect, and as a result, have found that exosomes derived from a conditioned medium of neural stem cells extracted from the ventricular zone of the human brain exhibits an atopy inhibitory effect while containing a number of anti-inflammatory factors, thereby completing the present invention.

### DISCLOSURE OF INVENTION

### Technical Problem

It is an object of the present invention to provide a pharmaceutical composition for treating atopy, which contains stem cell-derived exosomes and has an excellent atopy inhibitory effect, and a method of treating atopy by administering the composition.

### Technical Solution

To achieve the above object, the present invention provides a pharmaceutical composition for treating atopy, which contains, as an active ingredient, neural stem cell-derived exosomes or a neural stem cell-conditioned medium containing neural stem cell-derived exosomes.

The present invention also provides a method for treating atopy, the method comprising administering neural stem cell-derived exosomes or a neural stem cell-conditioned medium containing neural stem cell-derived exosomes.

The present invention also provides a method for alleviating atopic dermatitis, the method comprising topically applying to affected skin areas neural stem cell-derived exosomes or a neural stem cell-conditioned medium containing neural stem cell-derived exosomes.

The present invention also provides a functional cosmetic composition for alleviating atopic dermatitis comprising neural stem cell-derived exosomes or a neural stem cell-conditioned medium containing neural stem cell-derived exosomes.

### Advantageous Effects

The present invention is effective for the treatment of atopic dermatitis, unlike steroids and immunomodulators as conventional therapeutic agents against atopic dermatitis, which adversely affects the growth and development of children and has a side effect of causing a burning sensation on the skin when used.

### BRIEF DESCRIPTION OF THE DRAWINGS

FIG. 1 schematically shows a method of extracting extracellular vesicles from a neural stem cell-conditioned medium, in which the method comprises isolating only exosomes having a size of 50 to 150 nm by centrifugation and liquid column chromatography using a column.

FIGS. 2A and 2B show the results of performing DLS (dynamic light scattering) analysis and NTA (nanoparticle tracking analysis) to determine the size of isolated extracellular vesicles corresponding to fractions 6 to 10 presumed to be exosomes; FIG. 2C shows the results of Western blotting performed to confirm the expression of CD63 and CD9 known as exosome markers; and FIG. 2D is a TEM (transmission electronic microscope) image showing cup-shaped vesicles having the characteristic shape of vesicles such as exosomes.

FIGS. 3A and 3B show the results of real-time qPCR, which indicate that inflammatory response induced by TNFα and IFNγ was inhibited when the HaCaT cell line was treated with a neural stem cell-conditioned medium, and show that inflammatory cytokines IL-6 and TNFα decreased, and FIGS. 3C, 3D and 3E show that the expression of inflammatory chemokines decreased.

FIG. 4A shows the results of observing changes in the shape of the RAW264.7 cell line treated with a neural stem cell-conditioned medium, and demonstrates that treatment with the neural stem cell-conditioned medium alone causes no inflammation; FIG. 4B shows that the neural stem cell-conditioned medium decreased the LPS-induced expression of inflammatory cytokine IL-6 and COX-2 in the RAW264.7 cell line; FIG. 4C shows that the expression of the iNOS (induced nitric oxide synthase) gene which is involved in the production of nitric oxide (NO), a reactive oxygen species; and NO produced by iNOS was decreased by treatment with the neural stem cell-conditioned medium.

FIG. 5A shows the results of Western blot analysis, which indicate that when inflammation was induced by TNFα and IFNγ after treating HaCaT with a neural stem cell-conditioned medium, the protein expression level of inflammatory cytokine IL-1β decreased in a manner dependent on the concentration of the conditioned medium; and FIG. 5B shows that the results of Western blot analysis, which indicate that phosphorylation of the transcription factor NF-kB that regulates the expression of inflammatory cytokines and chemokines decreased.

FIG. 6 shows the results of analyzing the expression level of inflammatory cytokine TNFα after treating HaCaT cells with exosomes isolated from a neural stem cell-conditioned medium in order to examine whether the exosomes would inhibit inflammatory response induced by TNFα and IFNγ.

FIGS. 7A and 7B show the results of examining whether the expression of chemokines involved in the activation and infiltration of T-cells and macrophages were decreased by exosomes.

FIG. 8A shows the results of ICC (immunocytochemistry), which indicate that when HaCaT cells were treated with neural stem cell-derived exosomes stained with the green fluorescent dye PKH67, the exosomes were internalized into the cells, and

FIG. 8B shows the results of Western blotting, which indicate that phosphorylation of the transcription factor NF-kB that regulates the expression of inflammatory cytokines decreased after treatment with the exosomes.

FIG. 9 schematically shows an experimental method in which NC/NgA mice were treated with each of NSC-CM (neural stem cell-conditioned medium), SC-Exo (neural stem cell-conditioned medium-derived exosomes) and tacrolimus.

FIG. 10A shows the skin of NC/NgA mice after treating the mice with each of NSC-CM (neural stem cell-conditioned medium), NSC-Exo (neural stem cell-conditioned medium-derived exosomes) and tacrolimus for 21 days and FIG. 10B shows the enlarged picture of NSC-Exo.

FIG. 11 shows the results of H & E staining of skin tissues obtained by sacrificing NC/NgA mice treated with each of NSC-CM (neural stem cell-conditioned medium), SC-Exo (neural stem cell-conditioned medium-derived exosomes) and tacrolimus for 21 days.

FIG. 12 shows results indicating that IgE expression in mouse serum was decreased by treating NC/NgA mice with the atopy inducer DNCB (dinitrochlorobenzene) and then treating the mice with NSC-Exo (neural stem cell-conditioned medium-derived exosomes).

FIG. 13A shows the results of toluidine blue O staining of the skin tissues obtained NC/NgA mice treated with each of NSC-CM (neural stem cell-conditioned medium), SC-Exo (neural stem cell-conditioned medium-derived exosomes) and tacrolimus for 21 days; and FIG. 13B is a quantitative representation of the staining result in FIG. 13A.

FIG. 14 shows the results of selecting innate immune response-related proteins using the DAVID v6.8 program after LC-MS analysis of proteins in exosomes isolated from neural stem cell-conditioned medium.

### BEST MODE FOR CARRYING OUT THE INVENTION

Unless defined otherwise, all technical and scientific terms used herein have the same meanings as appreciated by those skilled in the field to which the present invention pertains. In general, the nomenclature used herein is well-known in the art and is ordinarily used.

The present invention is intended to develop an atopic dermatitis therapeutic agent based on stem cells using extracellular vesicles (exosomes), which exactly reflect the immunosuppressive properties and characteristics of neural stem cells, and a conditioned medium containing the exosomes, by culturing neural stem cells present in the human ventricular zone rather than culturing cord blood, bone marrow and adipose-derived stem cells.

Technologies of isolating mesenchymal stem cell-derived exosomes, which may be used against inflammatory diseases, various adult tissues, are known, the anti-inflammatory effect or atopy alleviation effect of exosomes obtained from human neural stem cells is not yet known. In particular, proteomics analysis demonstrated that exosomes derived from neural stem cell-conditioned medium, which are used in the present invention, contain a number of anti-inflammatory factors, and these factors exhibit an atopy inhibitory effect.

Therefore, in one aspect, the present invention is directed to a pharmaceutical composition for treating atopy, which contains, as an active ingredient, neural stem cell-derived exosomes or a neural stem cell-conditioned medium containing neural stem cell-derived exosomes.

In another aspect, the present invention is directed to a method for treating atopy, the method comprising administering neural stem cell-derived exosomes or a neural stem cell-conditioned medium containing neural stem cell-derived exosomes.

In the present invention, the exosomes may have a size of 50 to 150 nm, and may be CD63 and CD9 positive.

In the present invention, the neural stem cells may be cells derived from a ventricular zone, more preferably stem cells obtained by immortalizing cells derived from a ventricular zone of a fetal brain.

The present invention has been conceived to solve the above-described problems using neural stem cell-derived exosomes and a conditioned medium containing the exosomes, which are systems that carry anti-inflammatory factors capable of immunomodulation and immunosuppression.

In an example of the present invention, it was confirmed that a conditioned medium of human ectodermal neural stem cells and exosomes extracted from the conditioned medium contained various anti-inflammatory factors and cytokines, and thus inhibited excessive inflammatory response induced by TNFα (tumor necrosis factor alpha) and IFNγ (interferon gamma). In particular, it was confirmed that the conditioned medium and the exosomes inhibited the phosphorylation of NF-κB which is involved directly in inflammatory response, thereby ultimately inhibiting the expression of inflammatory cytokines. In addition, in view of application, an application method of transplanting stem cells directly into the skin or injecting stem cells intravenously has adverse effects, such as pulmonary embolism or the possibility of differentiation into other cells, but the use of a conditioned medium containing the active ingredients of stem cells and exosomes extracted from the conditioned medium has an advantage in that the conditioned medium and the exosomes may be easily applied.

In another example of the present invention, it was confirmed that when NC/NgA mice, which are atopic dermatitis mouse models, were treated with each of a conditioned medium of neural stem cells, exosomes extracted from the conditioned medium, and tacrolimus as a positive control for 21 days, skin slough and dry skin in the groups treated with each of the neural stem cell-conditioned medium, the exosomes derived therefrom and tacrolimus were alleviated and the skin thickness in these groups was thinner close to the normal thickness.

In an example of the present invention, proteins in exosomes isolated from a neural stem cell-conditioned medium were analyzed using TMT (tandem mass tag) mass spectrometry, and annexin 1, isoform 2 of clusterin, isoform 2 of N-acetylmuramoyl-L-alanine amidase, and transcription intermediary factor 1-beta were selected as immune response inhibitors involved in anti-inflammation in the exosomes.

In a preferred embodiment, the pharmaceutical composition according to the present invention may be prepared in the form of an aqueous solution for parenteral administration. Preferably, a physically appropriate buffer, such as Hank's solution, Ringer's solution, or physically buffered saline, may be used. Water-soluble injectable suspensions may include a substrate that can increase the viscosity of the suspension, such as sodium carboxymethyl cellulose, sorbitol, or dextran. Additionally, suspensions of the active ingredient may be prepared as suitable oily injection suspensions. Suitable lipophilic solvents or vehicles include fatty oils such as sesame oil, or synthetic fatty acid esters, such as ethyl oleate or triglycerides, or liposomes. Polycationic amino polymers may also be used as carriers. The suspension may optionally contain suitable stabilizers or agents, which increase the solubility of the compounds to allow for the preparation of highly concentrated solutions.

The therapeutic composition according to the present invention may further contain a pharmaceutically acceptable carrier, and the pharmaceutically acceptable carrier includes a carrier, an adjuvant and a vehicle, which are collectively referred to as “pharmaceutically acceptable carriers”. The pharmaceutically acceptable carrier that can be used for the pharmaceutical composition of the present invention may include, but is not limited to, ion exchange resins, alumina, aluminum stearate, lecithin, serum proteins (e.g., human serum albumin), buffer substances (e.g., partial glyceride mixtures of several phosphates, glycine, sorbic acid, potassium sorbate and saturated vegetable fatty acids), water, salts or electrolytes (e.g., protamine sulfate, disodium hydrogen phosphate, potassium hydrogen phosphate, sodium chloride and zinc salts), colloidal silica, magnesium trisilicate, polyvinyl pyrrolidone, cellulose-based substrates, polyethylene glycols, sodium carboxymethylcellulose, polyarylates, waxes, polyethylene-polyoxypropylene-blocking polymers, polyethylene glycols, wool and the like.

As used herein, the term “prevention” refers to any action that prevents or delays atopy by administration of the composition of the present invention, and, as used herein, the term “treatment” refers to any action that improves or positively alters the symptoms of atopy by the administration of the composition of the present invention. The atopy treatment is applicable to any mammal that may be afflicted with atopy, including, but not limited to, humans and primates, as well as domestic animals such as cattle, pigs, sheep, horses, dogs and cats, but preferably humans.

As used herein, the term “administration” refers to an action of introducing the pharmaceutical composition according to the present invention into a subject by any appropriate method, and the route of administration of the composition may be any general route, so long as it enables the composition to be delivered to a target tissue. The pharmaceutical composition may be administered intraperitoneally, intravenously, intramuscularly, subcutaneously, intradermally, orally, topically, intranasally, intrapulmonarily or rectally, but is not limited thereto. Upon oral administration, peptides are digested, so that an oral composition may be coated with an active drug or may be formulated so as to protect the same from degradation in the stomach.

In addition, the pharmaceutical composition of the present invention is determined according to the type of drug, which is the active ingredient, as well as various related factors such as the disease to be treated, the route of administration, the age, gender and body weight of the patient, and the severity of the disease.

In still another aspect, the present invention is directed to an external skin preparation for alleviating atopic dermatitis, which contains, as an active ingredient, neural stem cell-derived exosomes or a neural stem cell-conditioned medium containing neural stem cell-derived exosomes.

In still another aspect, the present invention is directed to a method for alleviating atopic dermatitis, the method comprising topically applying to affected skin areas neural stem cell-derived exosomes or a neural stem cell-conditioned medium containing neural stem cell-derived exosomes.

In still another aspect, the present invention is directed to a functional cosmetic composition for alleviating atopic dermatitis, which contains, as an active ingredient, neural stem cell-derived exosomes or a neural stem cell-conditioned medium containing neural stem cell-derived exosomes.

Depending upon its function, the cosmetic compositions of the present invention may be provided in various forms, such as solutions (lotion type compositions), thickened solutions, gels, ointments, emulsions (cream, milks), vesicular dispersions, powders, dense powders, pastes or solid agents. More specifically, the cosmetic compositions of the present invention may be dispersed in various forms, including, but not limited to, blushers, creams (including face creams, hand creams, moisturizing creams and sunscreen creams), cream powders, eye liners, eye shadows, eyebrow pencils, foundations, lotions, mascaras, microemulsions, ointments, pomades and rouges. They may also be packaged in pressure packs containing a propelling agent permitting application in the form of foams or sprays.

Oral administration of the pharmaceutical composition according to the present invention is particularly useful when the desired treatment relates to a site or organ that is easily accessible by topical application. When applied topically to the skin, the pharmaceutical composition should be formulated in a suitable ointment containing the active ingredient suspended or dissolved in a carrier. The carrier for topical administration of the compound of the present invention includes, but is not limited to, mineral oil, liquid paraffin, white petrolatum, propylene glycol, polyoxyethylene, polyoxypropylene compounds, emulsifying wax and water. Alternatively, the pharmaceutical composition may be formulated in a suitable lotion or cream containing the active compound suspended or dissolved in a carrier. The suitable carrier includes, but is not limited to, mineral oil, sorbitan monostearate, polysorbate 60, cetyl ester wax, cetearyl alcohol, 2-octyldodecanol, benzyl alcohol and water. The pharmaceutical composition of the invention can also be applied topically in the form of a rectal suppository or suitable enema to the lower intestine. Topically applied transdermal patches also fall within the scope of the present invention.

The exosomes or neural stem cell-conditioned medium described herein may be added to various type of cosmetic compositions. For example, they may be added to pharmaceutical compositions which protect the human epidermis, hair and mucous membranes; makeup compositions for the skin and superficial body growths; compositions for buccodental use such as dentifrices; or ophthalmic compositions such as collegria, and the like.

The cosmetic compositions may contain natural or synthetic waxes. Natural waxes may be of animal origins, such as lanolin, beeswax, spermaceti or lanolin derivatives, such as lanolin alcohols, hydrogenated or acetylated lanolin, fatty acids of lanolin or acetylated lanolin alcohol, or of vegetable origin, such as carnauba, candelilla, kapok, rice, hydrogenated jojoba, alfa or yapan wax or cork fiber, sugar cane waxes, cocoa butter or the like. Alternatively, mineral waxes may include paraffin, montan, lignite, petrolatum, petrolatum waxes or microcrystalline waxes, ceresin, or ozokerite. Examples of synthetic waxes that can be used in the present invention include polyethylene waxes, the waxes obtained by Fischer-Tropsch synthesis and the linear esters resulting from the reaction of a saturated C₁₀-C₄₀ carboxylic acid and of a saturated C₁₀-C₄₀ alcohol, such as myristyl myristate. Other waxes that can be used in the present invention include calcium lanolates or stearates, or hydrogenated coconut oil, and the like.

The cosmetic composition may contain modified or unmodified oils of vegetable or animal origin, for example, sweet almond oil, avocado oil, castor oil, olive oil, jojoba oil, sunflower oil, wheat germ oil, sesame oil, groundnut oil, grapeseed oil, soybean oil, safflower oil, coconut oil, maize oil, hazelnut oil, karite butter, palm oil, apricot kernel oil, calophyllum oil or perhydrosqualine. Moreover, the oil phase may be a mineral oil, for example, liquid paraffin, liquid petrolatum and the like. The oil may be a synthetic oil, e.g. fatty acid esters, such as isopropyl myristate, isopropyl palmitate, 2-ethylhexyl palmitate, penicillin oil (stearyl octonate), unsaturated fatty acids, such as oleic, palmetic, stearic, behenic, linoleic, lanoleic acid or volatile or non-volatile isoparaffins, e.g. C₈-C₁₆ isoparaffins, and the like.

Moreover, the oil may be C₁₂-C₁₈ fatty alcohols, such as oleyl alcohol, cetyl alcohol and stearyl alcohol and the like.

If present an emulsion, the emulsified composition of the present invention comprises an oil phase and an aqueous phase. The oil phase is preferably used in an amount ranging from about 1% to about 75% by weight, more preferably from about 5% to about 60% by weight, most preferably from about 40% to about 60% by weight, based on the total weight of the composition.

The aqueous phase may include adjuvants commonly used in aqueous gels and cosmetic emulsions. The aqueous phase may be used in an amount ranging from about 0.5% to about 20% by weight, based on the total weight of the composition and may also include a lower C₂-C₆ monoalcohol and/or a polyol, such as glycerol, butylene glycol, isoprene glycol, propylene glycol, ethylene glycol, and the like.

Emulsifiers can be used to assist in preparing an emulsified cosmetic composition. Any amount of cosmetically acceptable emulsifier can be used as long as it exhibits the desired emulsifying effect. The emulsifiers are generally selected from known soaps and surfactants. Preferably, the emulsifiers are selected from stearic acid, sorbitan sesquinoleate, polyethylene glycol (PEG-30), dipolyhydroxystearate, lecithin, magnesium stearate, and derivatives and mixtures thereof. The emulsifiers preferably are used in amounts ranging from about 0.5 to about 30% by weight, more preferably from about 1% to about 12% by weight, even more preferably from about 4% to about 8% by weight, based on the total weight of the composition.

A thickener may also be used in the present invention. Any thickening agent normally used in cosmetics can be used. Examples of the thickener include modified clays, such as modified magnesium silicate (bentone gel VS38 from Rheox), hectoxite modified by distearyldimethylammonium chloride (benetone 38 CE from Rheox), cross-linked polyacrylic acids and guar gums and celluloses which may or may not be modified.

The composition of the present invention may comprise a film-forming compound. The film forming compound can be chosen from polymers in aqueous dispersions, such as, for example, acrylic, polyester and/or polyurethane polymers in aqueous dispersion, e.g. partially neutralized vinyl acetate/vinyl p-tert-butyl/benzoate/crotonic acid copolymer in aqueous dispersion.

The composition of the present invention may also comprise a coloring agent. The coloring agents may be either inorganic or organic pigments which are insoluble in aqueous and organic media, or dyes which are soluble in aqueous or organic matter.

A lubricating agent is another optional component. A lubricating agent generally aids in creating a soft and smooth feel of the composition to the hands. Examples of the lubricating agent include benzoic acid esters of C₁₂-C₁₅ alcohols, volatile silicones such as cyclomethicones, dimethicones and derivatives thereof. Examples of silicon-containing compounds useful in the present invention include cyclomethicone tetramer and pentamer (available as Dow Corning 244 or 245 Fluids) or non-volatile silicones such as stearyl dimethicone (available as Dow Corning 2503 Cosmetic wax), or derivatives thereof, such as cyclomethiconepolyol, dimethiconepolyol, cetyl dimethicone copolyol, phenyl methicone, phenyl trimethicone, and the like. Those skilled in the art will also appreciate that the silicon-containing compounds may also function as emulsifiers.

The silicon-containing compound is preferably used in an amount ranging from about 1% to about 50% by weight, more preferably from about 5% to about 30% by weight, most preferably from about 10% to about 25% by weight, based on the total weight of the composition.

A moisturizer may also be added to the cosmetic composition. A “moisturizer” refers to an agent that provides a moisturizing effect to the skin, such as a humectant.

The moisturizer is present in a moisturizing effective amount. Examples of the moisturizer include glycerin, butylene glycol, propylene glycol, sorbitol, sodium PCA, glucam E-10, glucam E-20, and the like. Preferably the moisturizer is used in an amount ranging from about 0.1% to about 10% by weight, more preferably from about 0.1 to about 5% by weight, most preferably from about 1% to about 5% by weight, based on the total weight of the composition.

Additionally, an emollient may be included as needed. It is preferred that the composition contains one or more emollients. The emollient is used to impart a smooth and soft feeling to the skin surface. This is effected without measurably affecting the skin hydration level and/or the skin liquid barrier. Examples of the emollient include vegetable triglycerides, such as avocado oil, olive oil, sunflower oil, organic acid esters such as sorbitan oleate, myristyl myristate, isopropyl myristate, glycol oleate, mineral oil, glycerin, petrolatum, petroleum jelly, and the like. The emollient is preferably used in an amount ranging from about 1% to about 50% by weight, more preferably from about 5% to about 40% by weight, most preferably from about 10% to about 25% by weight, based on the total weight of the composition.

Additionally, an antioxidant may also be included in the cosmetic composition of the present invention. The antioxidant may be natural or synthetic. Examples of the antioxidant include tocopherol, vitamin E derivatives, e.g., vitamin E linoleate, Vitamin E, vitamin E POE succinate, vitamin E acetate, ascorbic acid, ascorbyl palmitate and ascorbyl-PMG. It is used in effective amounts to neutralize harmful oxidants, e.g. singlet oxygen. It is preferably used in an amount ranging from about 0.1% to about 5% by weight, more preferably from about 0.5% to 1% by weight, based on the total weight of the composition.

The cosmetic composition may also contain ingredients that reduce the oily feel of emollients in the composition, e.g., cream, known in the art, e.g., PPG-2-myristyl ether propinate, isopropyl palmitate and the like. If present, they are preferably contained in an amount ranging from about 1% to about 30% by wieght, more preferably from about 5% to about 25% by wieght, most preferably from about 10 to about 20% by weigh, based on the total weight of the composition.

A pH adjusting agent is commonly used to adjust the acidity of the composition to a desirable range, preferably to a pH of about 6 to 8.

An example of the pH adjusting agent is aminomethylpropanol. This pH adjusting agent is added in an amount effective to change the pH of the composition to the desired pH range.

Preferably, the pH adjusting agent is used in an amount of less than 0.4% by weight, based on the total weight of the composition.

A sunscreen agent may be contained as needed. The expression “sunscreen agent” denotes sunscreen agents that are useful in absorbing, screening or preventing ultraviolet rays from penetrating the skin. Preferably, the sunscreen agent is titanium dioxide or zinc oxide, and more preferably the sunscreen agent is coated with a polymeric material or other cosmetically acceptable coating. Examples of sunscreen agents include micronized titanium dioxide coated with aluminum stearate, or C₉-C₁₅ polyfluoroalkyl phosphate, polymer coated zinc oxide, aminobenzoic acid (PABA) and its esters, benzophenone-3, octyl salicylate, menthyl anthranilate, phenylbenzimidazole sulfonic acid and the like.

The balance of the composition is comprised of customary additives selected from auxiliaries, fillers, organic solvents (such as alcohols and oils), buffers, perfumes, self-tanning agents (e.g. dihydroxy acetone) and the like and other cosmetically acceptable carriers and fillers. Other conventional additives, that can be used in the cosmetic composition, include dispersants and preservatives. The additives are typically used in an amount of ranging from about 0% to about 20% by weight, preferably from about 1% to about 15% by weight, more preferably from about 4% to about 10% by weight, based on the total weight of the composition.

It will be appreciated that the remaining percentage or balance of the composition is water. Water acts as a vehicle to ensure even distribution of the composition to the skin. It is preferred that the water used in the present invention is deionized or distilled water and the water is typically used in an amount ranging from about 10% to about 80% by weight, most preferably from about 20% to about 40% by weight, based on the total weight of the composition.

In a preferred embodiment, the cosmetic composition of the present invention is a cream or lotion or ointment, but most preferably a cream. The carrier is preferably water. The preferred cream composition is formulated as an oil in water emulsion that contains the necessary common cosmetic ingredients known to one skilled in the art for ensuring high user properties, in addition to the exosome or neural stem cell conditioned medium component. In one preferred embodiment, these ingredients preferably include petrolatum, especially white petrolatum, in an amount ranging from about 0.5% to about 1% by weight of the composition, lanolin alcohol preferably in an amount ranging from about 0.5% to about 1% by weight of the composition, PPG-2-myristyl ether propionate in an amount ranging from about 3% to about 6% by weight of the composition, mineral oil in an amount ranging from about 5% to about 10% by weight of the composition, triethanolamine in an amount ranging from about 0.2% to about 0.8% by weight of the composition, glycerol, particularly distilled glycero in an amount ranging from about 2% to about 4% by weight of the composition, stearic acid in an amount ranging from about 15% to about 20% by weight of the composition, isopropyl palmitate in an amount ranging from about 10% to about 15% by weight of the composition, and the remainder is water. The cosmetic or pharmaceutical composition of the present invention may comprise fragrances, perfumes, vitamins and other common ingredients known to one of ordinary skill in the art, as well as antioxidants and coloring agents, in addition to those described hereinabove, depending on the specific cream formulation desired.

### EXAMPLES

Hereinafter, the present invention will be described in further detail with reference to examples. It will be obvious to a person having ordinary skill in the art that these examples are for illustrative purposes only and are not to be construed to limit the scope of the present invention.

### Example 1: Preparation of Neural Stem Cell-Conditioned Medium

A cell line was obtained by immortalizing adult neural stem cells (NSCs) isolated from a ventricular zone of a fetal brain. 14-week-old fetal neural cell tissue was separated into single cells by treatment with a solution containing 0.1% collagenase and 0.1% hyaluronidase at 37° C. for 1 hour and treatment with 0.05% trypsin-EDTA for 2 to 3 minutes, and then neural stem cells were isolated therefrom by FACS using neural stem cell-specific markers (CD45−/CD133+/CD34−). The cells were cultured in human neurosphere culture medium containing N2 supplements, 0.2 mg/ml heparin, 20 ng/ml bFGF (basic fibroblast growth factors), 20 ng/ml EGF (epidermal growth factor) and 10 ng/ml LIF. After 10 to 14 days, the formed neurospheres were separated into single cells by treatment with collagenase, and v-myc gene was transduced into the cells by a retroviral vector, followed by antibiotic screening. The resulting cells were cultured in non-inducing medium containing DMEM (Dulbecco's Modified Eagle Medium), 10% FBS (fetal bovine serum) and 1% penicillin/streptomycin (Flax J D et al., Nature Biotechnology, vol. 16, 1998; Lim H-C et al., Neuroscience Letters 435, pp 175-180, 2008). The cells were dispensed into a 150 mm culture dish at a density of 5×10⁵ cells and 15 ml of culture medium was added thereto, and then the culture medium was collected at a confluence of 80% in an incubator at 37° C. under 5% CO₂. At this time, the culture medium was a non-inducing medium containing DMEM, 10% FBS and 1% penicillin/streptomycin, and after the culture, non-adherent cells were removed. During subculture of the neural stem cells cultured through the above-described process, the conditioned medium was centrifuged and the supernatant was collected by filtration.

### Example 2: Isolation of Neural Stem Cell-Derived Exosomes

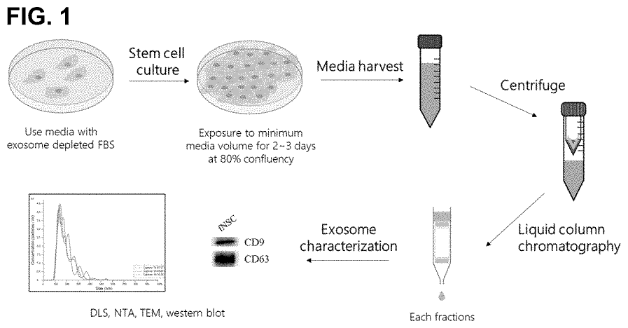

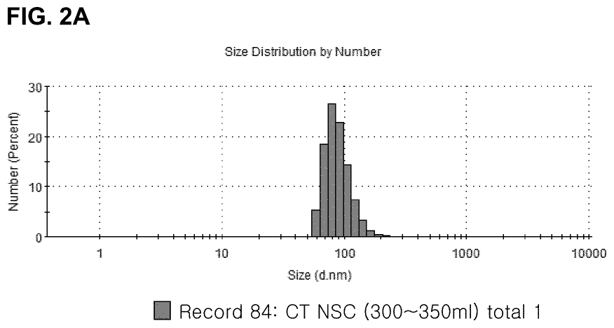

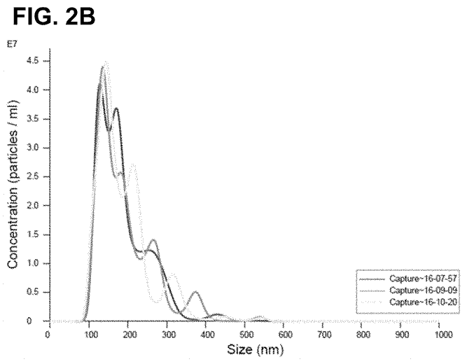

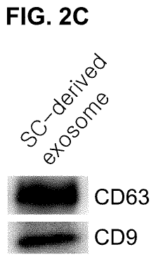

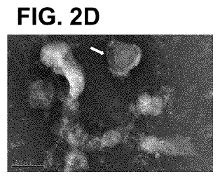

The immortalized neural stem cells obtained in Example 1 were dispensed in a 150 mm culture dish at a density of 5×10⁵ and cultured. When a confluence of 80% was reached, 13. 5 ml of culture medium was added and the cells were cultured in an incubator at 37° C. under 5% CO₂ incubator for 2 days, and then the culture medium was collected. The collected culture medium was centrifuged at 10,000 g for 30 minutes at 4° C. to remove cell debris. The remaining medium was filtered through a 0.22 μm bottle top filter, and concentrated by centrifugation using an Amicon 100K tube at 5,000 g and 4° C. for 15 minutes. The concentrated medium was applied to a column packed with Sepharose 2b beads at a maximum loading volume of 500 μl per application, and extracellular vesicles were collected according to size by a liquid column chromatography method. The column was sufficient washed twice with autoclaved PBS, and then 500 μl of the sample was applied to the column. 500 μl of a solution that passed through the column was considered as one fraction, and the sixth to tenth fractions were collected. Among the extracellular vesicles, extracellular vesicles having a size of 50 to 150 nm, called exosomes, were obtained (FIG. 1). The exosomes obtained through the above-described process were stored at −80° C. In order to confirm whether the isolated vesicles would be exosomes, the exosome-specific markers CD63 and CD9 were detected by Western blotting (FIG. 2C), and in order to confirm whether the vesicles would correspond to the size of exosomes, the size-dependent distribution and number of the vesicles were analyzed by DLS (dynamic light scattering) and NTA (nanoparticle tracking analysis) (FIGS. 2A and 2B). In addition, in order to confirm whether the vesicles would have a cup or doughnut shape which may be regarded as a specific shape of exosomes, the characteristic shape of the vesicles was confirmed by a transmission electron microscope (TEM) image (FIG. 2C).

### Example 3: Identification of Anti-Inflammatory Effect of Neural Stem Cell-Conditioned Medium

The human keratinocyte cell line HaCaT (ATCC) was seeded into a 12-well plate at a density of 2.5×10⁴ cells per well and treated with each of 10%, 50% and 100% neural stem cell-conditioned media for 48 hours. Then, the cells were treated with TNFα (10 ng/ml) and IFN-γ (10 ng/ml) for 6 hours to induce inflammation, and the cells were washed with PBS, and then harvested by treatment with trypsin-EDTA. Next, the anti-inflammatory effects of the neural stem cell-conditioned media and the exosomes derived from the neural stem cells were examined by real-time qPCR. When inflammation was induced with TNFα (10 ng/ml) and IFN-γ (10 ng/ml), the mRNA expression levels of the inflammatory cytokines IL-6 and TNFα and the chemokines TARC, RANTES and MCP-1 functioning as chemotaxis triggering immune cells were analyzed.

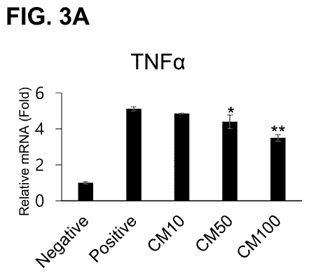

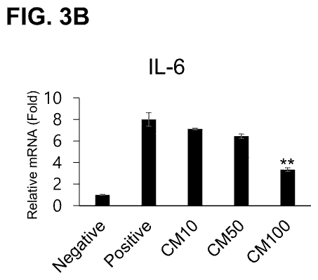

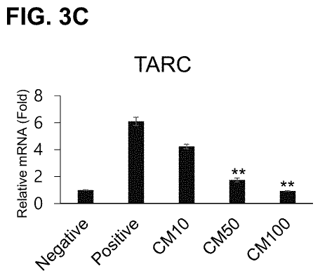

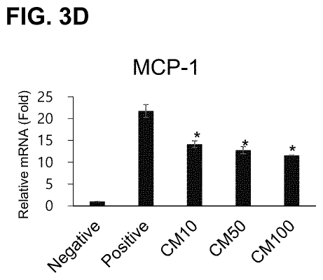

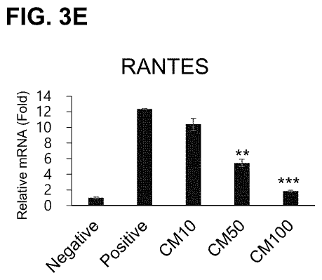

As a result, as can be seen in FIGS. 3A-3E, it could be confirmed that the expression levels of the inflammatory cytokines and chemokines were decreased by treatment with the neural stem cell-conditioned medium.

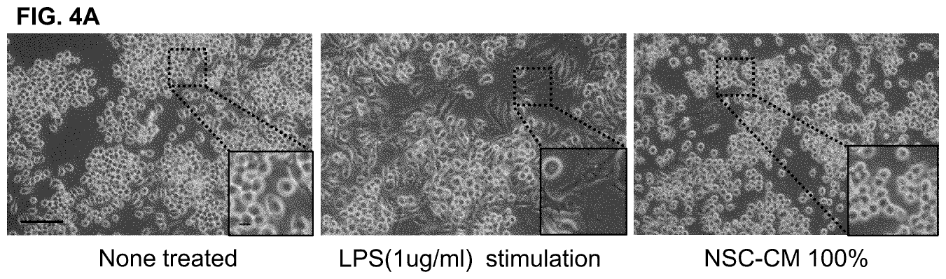

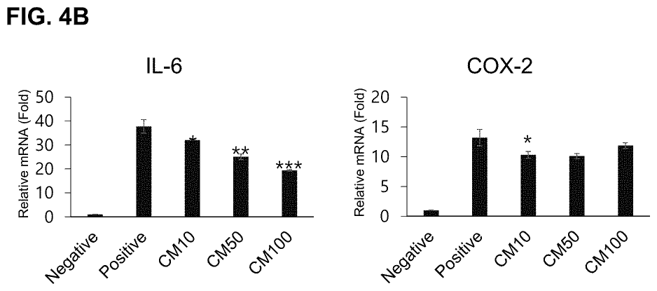

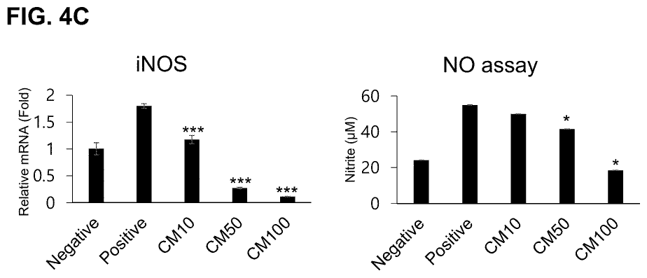

In addition, in order to confirm the anti-inflammatory effect of the neural stem cell-conditioned medium in immune cells, the mouse macrophage cell line RAW264.7 (Korean Cell Line Bank KCLB NO: 40071) was treated with each of 10%, 50% and 100% neural stem cell-conditioned media for 48 hours, and then treated with LPS (100 ng/ml) in the presence of the conditioned media to induce inflammation. Then, the anti-inflammatory effect of the conditioned media was examined by real-time qPCR. To measure the production of nitric oxide, the sample was incubated with Griess reagent at 1:1 (v/v), and then the absorbance at 548 nm was measured with a spectrophotometer. As can be seen in FIGS. 4B and 4C, the LPS-induced mRNA expression levels of IL-6 which is an inflammatory cytokine), COX-2 which is important in producing PGE2 that induces itching, and induced NOS (nitric oxide synthase) that produces the reactive oxygen species nitric oxide, and the concentration of nitric oxide which is the final product of iNOS, were decreased by treatment with the neural stem cell-conditioned media. In addition, as can be seen from the shape of macrophages, the neural stem cell-conditioned medium itself did not activate macrophages.

### Example 4: Identification of the Effects of Neural Stem Cell-Conditioned Medium on Inhibition of Phosphorylation of NF-kB (Nuclear Factor-Kappa B) Transcription Factor and Inhibition of Expression of IL-1β

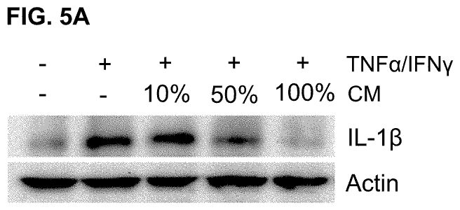

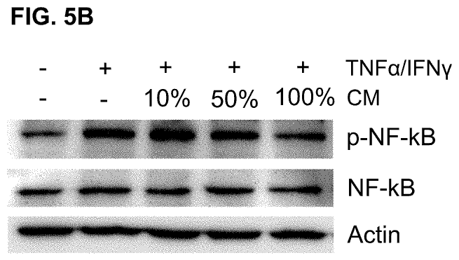

HaCaT cells treated with the neural stem cell-conditioned medium under the same conditions as described in Example 3 were treated with trypsin-EDTA, and proteins were collected from the cells by lysis buffer and quantified by Bradford assay. Using the proteins, the phosphorylation level of the transcription factor NF-kB that mediates inflammatory response was analyzed by Western blotting using NF-kB antibody and phospho-NF-kB antibody. In addition, the expression level of the mature form of IL-1β was examined using IL-1β antibody. As a result, as can be seen in FIGS. 5A and 5B, it could be seen that the phosphorylation level decreased in a manner dependent on the treatment concentration of the neural stem cell-conditioned medium.

### Example 5: Identification of Internalization of Exosomes Isolated from Neural Stem Cell-Conditioned Medium into HaCaT Cells

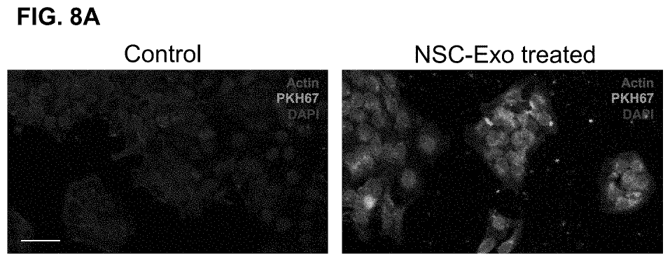

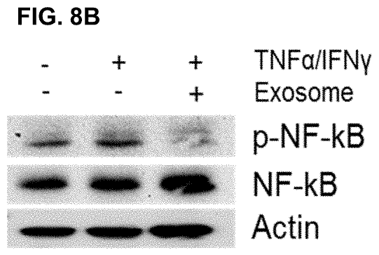

In order to stain the membrane of the exosomes with PKH67, a coverslip was applied onto a 24-well plate, and 1×10⁴ HaCaT cells were seeded thereon, and then treated with 1 μg/ml (on a protein concentration basis) of the exosomes for 24 hours. Then, the cells were fixed with 4% PFA (paraformaldehyde) overnight under a cold condition. After washing twice with PBS, the cells were treated with 0.1% Triton X-100 for 5 minutes so that intranuclear staining could be possible (FIG. 8A). After blocking with 10% NDS (normal donkey serum), the cells were treated with a 1:500 dilution of actin antibody in 2% NDS to stain cytoplasmic actin.

### Example 6: Identification of Anti-Inflammatory Effect of Exosomes Isolated from Neural Stem Cell-Conditioned Medium

HaCaT cells were seeded into a 12-well plate at a density of 2.5×10⁴ cells per well and exposed to 500 μg/ml of the isolated exosomes for 48 hours. Then, the cells were treated with TNFα (10 ng/ml) and IFN-γ (10 ng/ml) for 6 hours to induce inflammation. Next, the cells were washed with PBS, and then harvested by treatment with trypsin-EDTA, and the anti-inflammatory effect of the exosomes derived from the neural stem cells was examined by real-time qPCR.

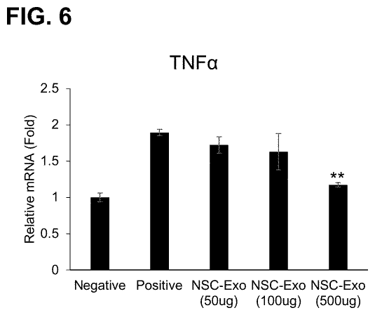

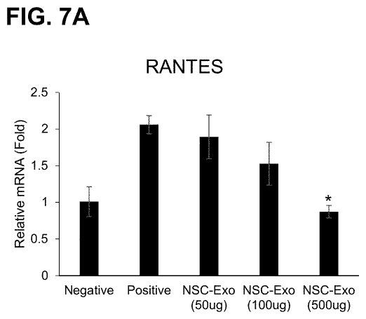

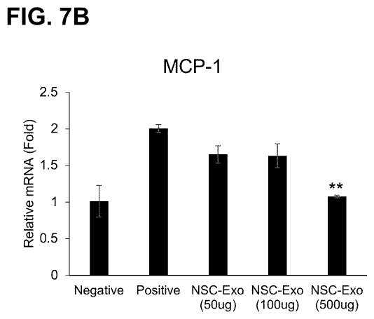

As a result, as shown in FIGS. 6 and 7A to 7C, it could be confirmed that the expression levels of the inflammatory cytokine TNFα and the chemokines TARC, MCP-1 and RANTES were decreased by treatment with the exosomes. In addition, the phosphorylation level of the transcription factor NF-kB (nuclear factor kappa B) involved in inflammatory response was analyzed by Western blotting under the same conditions as described above, and as a result, it could be confirmed that the protein expression of phosphorylated NF-kB was decreased by treatment with the exosomes (FIG. 8B).

### Example 7: Evaluation of Anti-Inflammatory Effects of Neural Stem Cell-Conditioned Medium and Exosomes Derived from Conditioned Medium in Atopic Dermatitis Mouse Model

NC/NgA mice (Charles river, imported by Orient) are atopic dermatitis disease model mice that show symptoms similar to those of human atopic dermatitis. The mice show no inflammation under SPF (specific pathogen-free) conditions, but show clinical symptoms similar to those of atopic dermatitis under conventional conditions and are suitable for an atopic dermatitis alleviation experiment. Thus, using the mice, an in vivo experiment was performed.

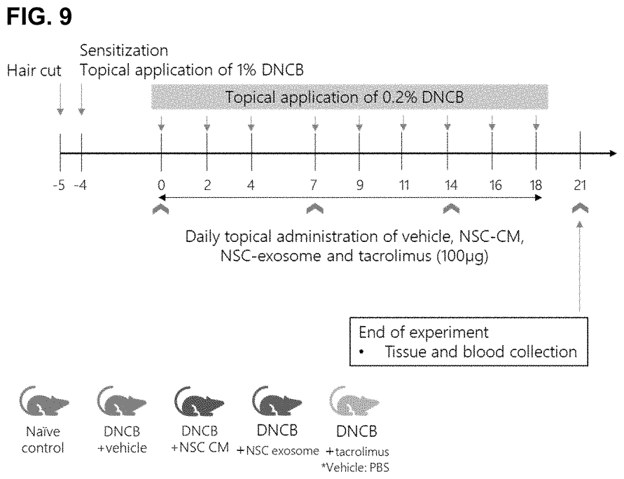

As shown in FIG. 9, twenty five 8-week-old male NC/NgA mice were housed in an SPF facility for 1 week, and on 5 days before the experiment started, hair in a 3 cm*4 cm area of the back of each animal was removed using hair removal cream and an animal hair shaver. Thereafter, the animals were divided into the following five groups, each consisting of five animals: negative; AD-induced; NSC-CM (neural stem cell-conditioned medium); NSC-Exo (neural stem cell conditioned medium-derived exosomes); and tacrolimus (Protopic ointment 0.03%, Astellas Pharma).

Here, tacrolimus is an immunosuppressive agent that is used against atopic dermatitis.

On 4 days before the start of the experiment, the groups other than the negative control group were sensitized by applying 200 μl of 1% DNCB (olive oil:acetone=1:3) to the skin of the back. From the day of start of the experiment, 0.2% DNCB (200 μl) was applied to the back once every two days to maintain atopic symptoms, and NSC-CM (100 μl), NSC-Exo (500 μl) and tacrolimus (100 μl) were applied to the affected parts of the respective groups each day for 21 days. At the end of the 21-day experiment, the affected skin tissues were collected and biopsied.

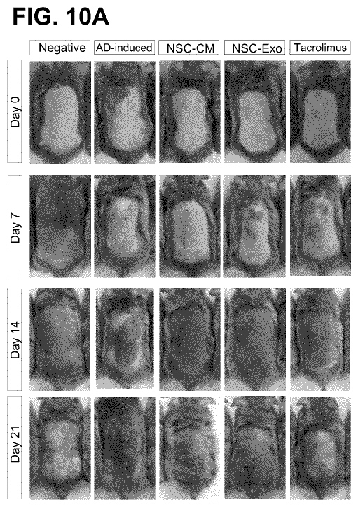

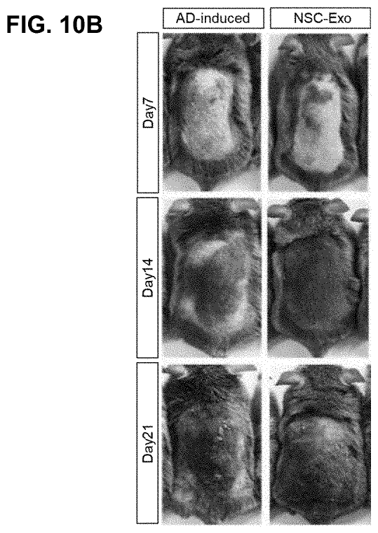

As a result, as shown in FIG. 10, it was confirmed that one week after the start of the experiment, in the atopic dermatitis-induced group, flaky skin and skin slough symptoms appeared, and after two weeks, in the groups treated with each of the neural stem cell-conditioned medium, the exosomes derived therefrom and tacrolimus, skin slough and dry skin were alleviated. In particular, as can be seen from the hair growth patterns, the hair grew more evenly in the groups treated with the test substance than in the atopic dermatitis-induced group, and in the atopic dermatitis-induced group, the skin got flaky, and thus the hair pulled out in lumps.

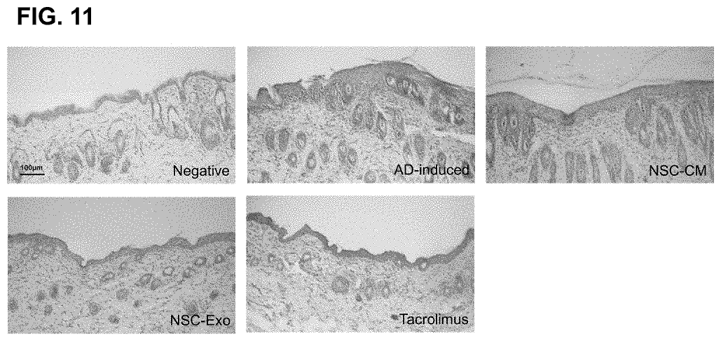

The skin tissues obtained by sacrificing the mice on day 21 were fixed in 4% para-formaldehyde and embedded in paraffin to make paraffin blocks. The paraffin blocks were cut to a size of about 7 μm and subjected to H & E staining. As a result, as shown in FIG. 11, it could be confirmed that in the atopic dermatitis-induced group, the epidermis became thickened due to inflammation and keratinocyte hyperplasia, but in the groups treated with each of the neural stem cell-conditioned medium, the exosome and tacrolimus, the thickened epidermis became thinner again.

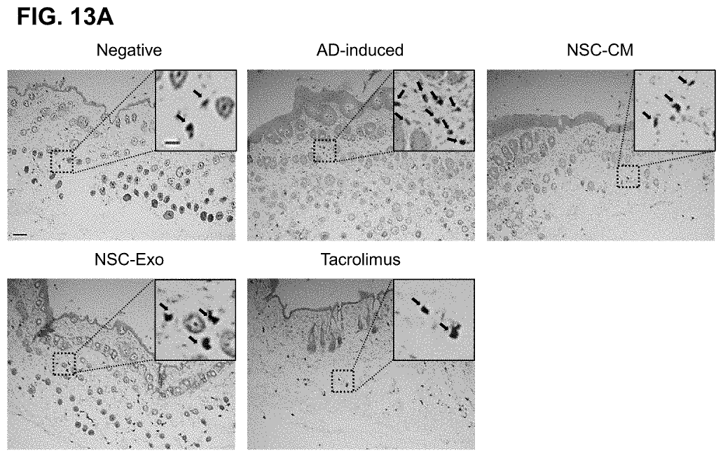

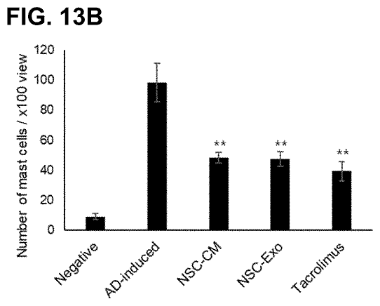

In addition, toluidine blue 0 staining was performed to confirm mast cell degranulation. When mast cells are degranulated by inflammatory cytokines, these secrete histamines, causing itching. As shown in FIG. 13, it could be visually seen that the mast cells stained with dark red purple were excessively infiltrated. On the other hand, it could be confirmed that in the groups treated with each of the neural stem cell-conditioned medium and the exosome, the infiltration of mast cells decreased.

### Example 8: Evaluation of the Anti-Inflammatory Effect of Exosomes Isolated from Neural Stem Cell-Conditioned Medium in Atopic Dermatitis Mouse Model

In order to evaluate the anti-inflammatory effect of the exosomes isolated from the neural stem cell-conditioned medium at the in vivo level, enzyme-linked immunosorbent assay (ELISA) capable of measuring an antibody response to a specific antigen was used. 8-week-old male NC/NgA mice to be used in the experiment were divided into the following three groups, each consisting of five mice: a normal mouse group; an atopic dermatitis-induced mouse group; and a mouse group treated with the neural stem cell-derived exosome). In the same manner as Example 7, the groups other than the negative control group (normal mouse group) were treated with 1% DNCB to induce atopic dermatitis. Blood was collected from the hearts of two mice randomly selected from each group, and serum was separated from the blood, and then the expression of mouse immunoglobulin E (IgE) in the serum was analyzed.

Specifically, using a KOMA ELISA kit (K3231082P), each of standard, control and serum was added to a pre-coated plate and incubated so as to be capable of reacting with the primary antibody of the plate, and was then reacted with secondary antibody after washing several times. The detection antibody made it possible to measure a signal by reaction with the enzyme-antibody-serum complex, and the antibody was labeled with a color development reagent (Pink-ONE TMB). Using the antibody, a color development reaction was performed at room temperature to a suitable level. When the color development proceeded sufficiently, the color development reaction was terminated using a stop solution, and finally, the absorbance at 450 nm was measured using a microplate reader.

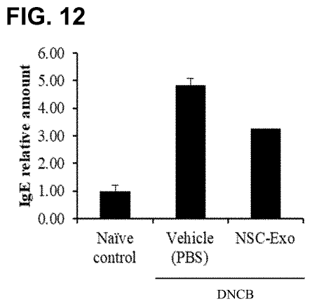

As a result, from a comparison between the normal mice (negative control) not treated with the atopic dermatitis inducer, the mice (vehicle) with the atopic dermatitis inducer DNCB, and the mice (NSC-Exo) treated with the atopic dermatitis inducer DNCB and the neural stem cell-derived exosome, the expression level of IgE was ⅓ lower in the mice (DNCB+NSC-Exo) treated with DNCB and the exosome than in the atopic dermatitis-induced mice (vehicle) (FIG. 12). This indicates that inflammatory response was alleviated by the neural stem cell-derived exosome.

### Example 9: Proteomics Analysis of Exosomes Isolated from Neural Stem Cell-Conditioned Medium

In order to analyze proteins in the exosomes isolated from the neural stem cell-conditioned medium in Example 2, TMT (tandem mass tag) mass spectrometry was used.

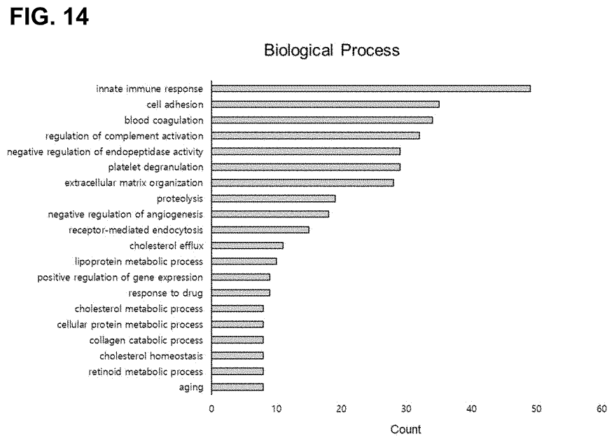

First, the membrane of the exosomes was broken using lysis buffer so that proteins in the exosomes were exposed. Then, the proteins were quantified using BCA (bicinchoninic acid) assay. The same amounts of the proteins were treated with trypsin/EDTA to make peptide units, and the peptides were reacted with isotope-labeled tandem mass tags. The prepared sample was fractionated into several portions by a high-pH fractionation method, and then analyzed by LC-MS (liquid chromatography mass spectrometry), and as a result, a total of 2685 proteins were detected. As shown in FIG. 14, 130 proteins related to innate immune response were selected using the DAVID (The Database for Annotation, Visualization and Integrated Discovery) v6.8 program. Among them, the number of peptides for qualitatively identifying specific proteins is referred to as PSMs (the number of peptide spectrum matches), and the peptides detected only at specific proteins are referred to as unique peptides. Proteins capable of being qualitatively and quantitatively identified, which had a PSMs value of 10 or more and a unique peptides value of 2 or more, were selected, and a protein list consisting of a total of 73 proteins was obtained. Finally, through Pubmed, Google Schola, UniPort and String websites, annexin 1, isoform 2 of clusterin, isoform 2 of N-acetylmuramoy-L-alanine amidase, and transcription intermediary factor 1 beta, related to immune response suppression, were finally selected.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A method for treating atopy, the method comprising administering neural stem cell-derived exosomes or a neural stem cell-conditioned medium containing neural stem cell-derived exosomes.

2. The method of claim 1, wherein the exosomes have a size of 50 to 150 nm.

3. The method of claim 1, wherein the exosomes are CD63 and CD9 positive.

4. The method of claim 1, wherein the neural stem cells are stem cells obtained by immortalizing cells derived from a ventricular zone of a fetal brain.

5. The method of claim 1, wherein the exosomes comprise annexin 1, isoform 2 of clusterin, isofom 2 of N-acetylmuramoy-L-alanine amidase, and transcription intermediary factor 1-beta.

6. A method for alleviating atopic dermatitis, the method comprising topically applying to affected skin areas neural stem cell-derived exosomes or a neural stem cell-conditioned medium containing neural stem cell-derived exosomes.

7. A functional cosmetic composition for alleviating atopic dermatitis comprising neural stem cell-derived exosomes or a neural stem cell-conditioned medium containing neural stem cell-derived exosomes.
